(19) United States
(12) Patent Application Publication
(10) Pub. No.: US 2013/0131684 A1
(43) Pub. Date: May 23, 2013

(54) BONE SCREW INTRODUCING SLEEVE

(71) Applicant: Eric D. Farrell, Los Angeles, CA (US)

(72) Inventor: Eric D. Farrell, Los Angeles, CA (US)

(21) Appl. No.: 13/699,463

(22) PCT Filed: May 25, 2011
(86) PCT No.: PCT/US11/37889

§ 371 Date: Feb. 6, 2013

Related U.S. Application Data
(60) Provisional application No. 61/348,499, filed on May 26, 2010.

(51) Int. Cl. A61B 17/86

(52) U.S. Cl. CPC A61B 17/86
USPC 606/104

(57) ABSTRACT

A guide sleeve that is deformable or splittable, for use in threading a surgical screw therethrough and into a drilled hole (24) at a surgical site for repair of a fractured bone. The guide sleeve has an inner diameter only as large as the screw shank, not its enlarged head, and the incision need only have a general diameter as large as the guide sleeve outer diameter, causing less trauma and permitting faster healing. A surgical drill may first be inserted through the guide sleeve for drilling the hole through the bone portions traversing the fracture. A surgical screw with enlarged head is then inserted into the guide sleeve to be threaded into the drilled hole, with the enlarged screw head temporarily and locally enlarging the guide sleeve as it passes therethrough until fully threaded into the drilled hole.

### CROSS REFERENCE TO RELATED APPLICATIONS

This application is a National Stage entry application of PCT International Patent Application No. PCT/US2011/37889 filed May 25, 2011, which claims the priority benefit of U.S. provisional patent application Ser. No. 61/348,499 filed May 26, 2010, the contents each of which are incorporated herein by reference in their entirety.

### FIELD OF THE INVENTION

This relates to the field of medical devices and more particularly to devices for orthopedic surgery.

### BACKGROUND OF THE INVENTION

Surgical plates and screws are known to be utilized to assist in maintaining the reduction of bone fractures in a patient, thus permitting those fractures to heal in their correct position. Conventionally, in order to place such a screw and/or plate (or, implant) at a selected site, an incision must be made through soft tissue to access the site. These incisions are generally as long as the plate itself. Any soft tissue between the skin and plate/bone must be cut or moved out of the way. This may lead to extensive soft-tissue dissection resulting in reduced blood supply to the fracture and subsequently delayed healing. A screw is placed through the plate and into bone by first drilling a hole into the bone with a drill bit. In general, the drill bit is inserted through a rigid tube which acts as a sleeve or guide in order to protect the surrounding soft tissues from being caught and wrapped into the drill bit as it spins.

Recently, “minimally invasive” techniques in fracture care are being utilized with great success. Fractures are reduced through closed means, and the plate is then inserted through a small incision distant from the fracture site and slid under the muscle and soft tissue directly on top of the bone. The screws are then inserted through small respective incisions, thus limiting soft-tissue injury. In minimally invasive surgery, locations of the screw holes are identified, or localized, with X-ray imaging (fluoroscopy), since direct visualization of the implants is not possible. Once a hole is localized, a small incision (approximately the size of the screw head) is made through the skin and a hemostat is used to spread the soft tissue over the plate hole. A rigid drill guide (tube or sleeve) is then placed through the soft tissue and into the hole in the plate, and its correct position is confirmed with fluoroscopy. A drill bit is then placed through the drill sleeve and drilled through the bone. The drill guide prevents the soft tissue from being wrapped around the drill bit as it spins. The drill guide must then be removed so that a depth gauge may be inserted to assure the proper depth of the drilled hole. The screw must then be inserted into the incision and advanced through the soft tissues to the hole in the plate and bone. Prior to advancing the screw into the bone, it may be necessary to verify screw position under fluoroscopy. The screw can then be threaded through the plate and bone. Since the screw is not surrounded by a sheath or guide, there is the potential for injury to the surrounding soft tissue during screw rotation while being inserted.

In U.S. Pat. No. 5,904,685 is disclosed a sheath having an inner protrusion cooperable with threading on the screw's shank to allow controlled threading into a drilled hole; the head of the screw is identical in diameter to the screw's shank. But, in many surgical screws, the screw head is wider than the screw's shank, and also is larger than the drilled hole, and therefore is larger than the drill bit and the rigid tube drill guide that surrounds and guides the drill bit during drilling.

If a sleeve is desired to facilitate controlled insertion of the screw into the drilled hole, such a sleeve conventionally is sufficiently large in inner diameter to permit passage therethrough of the screw's large head, thus requiring the incision and the surrounding soft tissue tract to be larger than that required for the drilling procedure. However, it is greatly desired to keep the size of the incision and soft-tissue tract to a minimum, while still holding the screw from outside the incision during insertion through the incision and throughout the screwing process. Furthermore, if a drill sleeve only is used, it must be removed prior to insertion of the screw. As the screw is inserted through the incision through the soft tissue, it can become dislodged from the screw driver and thus become “lost” in the soft tissue. This may require the use of fluoroscopy to retrieve the screw and/or enlarging the incision. One must also “find” the drill hole through tactile feel of the screw tip falling into the hole. Both of these issues have the potential for increased soft-tissue injury and complications, as well as for increasing the surgical time required.

In U.S. Pat. No. 6,857,343 and in U.S. Patent Publication No. US 2007/0005077, a driver instrument is disclosed wherein a screw is placed in a gripping distal end of the driver, and the screw is inserted through the incision to the drilled hole whereafter the screw is controllably threaded into the drilled hole. However, no sleeve is disclosed to be utilized through which the screw passes in order to pass through the incision.

It is currently difficult to maintain control of the orientation of the screw during the screwing process from outside of the incision unless a sleeve or screw guide is utilized. One way to control screw orientation would be to use another tube larger in diameter than the screw's head and therefore larger than the rigid tube used as a drill guide for the drill bit.

A two-part combined surgical drill and surgical screw guide is disclosed in U.S. Pat. No. 6,416,518: an outer common support cylinder or cannula includes a handle for manual manipulation thereof; and an inner cylindrical portion or drill guide is insertable into the outer cannula and is removable therefrom. The drill guide has a small inner diameter just sufficiently large to controllably retain the drill bit therethrough during drilling into the bone. The outer cannula includes an inner diameter that is sufficiently large to receive thereinto the drill guide, and that is also sufficiently large to receive the enlarged head of the surgical screw. After drilling is completed, the drill guide is removed from the outer cannula and the screw is then inserted into the outer cannula and is controllably passed therethrough and is threaded into the drilled hole. However, such an arrangement still requires a sufficiently large incision to contain the outer cannula.

It is desired to provide a screw guide that permits controlled passage therethrough of a screw having an enlarged head, while minimizing trauma to the patient. It is also desirable to reduce the number of steps required to complete the task, since this will lead to shorter operative time, which is directly related to patient outcomes.

### BRIEF SUMMARY OF THE INVENTION

Briefly, the present invention is a deformable, elastically stretchable (or splittable) guide sleeve through which the screw may be moved and thereafter be rotated to be screwed into the drilled hole. The enlarged screw head can temporarily stretch the sleeve as it passes therethrough, and consequently also momentarily stretch the walls of the soft tissue incision as the enlarged screw head passes therethrough, thus minimizing trauma to the patient. But control of the screw can be maintained, and an incision need not be formed or made to have a diameter as large as the diameter of the enlarged screw head in order to permit the screw to pass through the incision. The inventive guide sleeve may also be longitudinally perforated or scored to be splittable by the screw head.

The present invention is also directed to a method of using a deformable, elastically stretchable guide sleeve, wherein, in addition to inserting the surgical screw thereinto and therethrough into the drilled hole, the guide sleeve may initially surround the drill bit, optionally within a rigid tube drill guide, during drilling, whereafter the drill bit (and drill guide, if used) is removable therefrom for screw insertion. A depth gauge may also easily be used, insertable through the guide sleeve, to confirm the depth of the drilled hole, enabling re-insertion of the drill bit (or rigid tube drill guide) for additional drilling, if necessary. Additionally, the sleeve could accommodate a means for being gripped for ease of insertion, stabilization and removal, such as a handle. The sleeve may also have a radio-opaque marker to allow for ease of visualization with X-ray. The sleeve may be used in conjunction with an inner trocar which will stiffen the sleeve during insertion into the soft-tissue tract.

Alternatively, the sleeve may consist of a “composite” construction, wherein a portion(s) of the sleeve is made of a more rigid material, e.g., metal, such as surgical grade stainless steel; or, a stiffer plastic; and the remainder of the sleeve consist of the more pliable or deformable material. This would allow for added rigidity to the sleeve, if needed for deep surgical wounds, but still deform and “stretch” to allow passage of the screw head.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is an isometric view of a deformable guide sleeve of the present invention, also showing a surgical screw to be inserted into the guide sleeve after drilling has been performed;

FIG. 2 is an isometric view of an alternate embodiment of guide sleeve;

FIG. 3 is an elevation view of a bone with a fracture to be pinned, and an incision formed through soft tissue to access the site of the fracture; a sleeve of FIG. 1 has been placed through the incision to the bone site, and a drill bit inserted through the guide sleeve;

FIG. 4 is an elevation view similar to FIG. 3 in which the drill has been removed after drilling, and a depth gauge has been inserted through the guide sleeve;

FIG. 5 is an elevation view similar to FIGS. 3 and 4 in which the screw of FIG. 1 has been positioned to be inserted through a deformable guide sleeve within the incision at the site;

FIG. 6 is an elevation view similar to FIG. 5 in which the screw is being screwed into the drilled hole, with the enlarged screw head temporarily deforming/widening the deformable guide sleeve as the screw is pushed by a tool work end and rotated thereby; and

FIG. 7 is an elevation view similar to FIG. 6 in which the screw has been completely screwed into the bone, with the deformable sleeve in place.

### DETAILED DESCRIPTION OF THE INVENTION

In the drawings, like numerals indicate like elements throughout. Certain terminology is used herein for convenience only and is not to be taken as a limitation on the present invention. The terms “distal” and “proximal” refer, respectively, to directions closer to and away from the patient. The terminology includes the words specifically mentioned, derivatives thereof and words of similar import. The terms and expressions used herein, and the embodiments illustrated below, are not intended to be exhaustive or to limit the invention to the precise form disclosed. These terms, expressions and embodiments are chosen and described to best explain the principle of the invention and its application and practical use and to enable others skilled in the art to best utilize the invention.

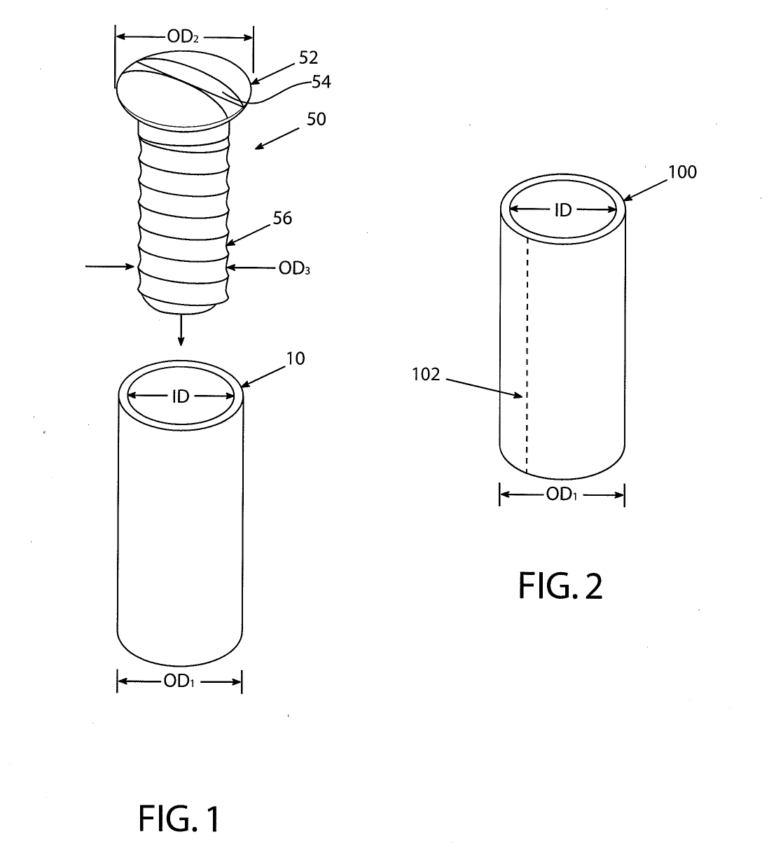

FIG. 1 depicts a deformable guide sleeve 10 of the present invention, for use in controlled insertion of a surgical screw 50 through an incision and into a hole drilled into a bone of a patient. Guide sleeve 10 is shown to have an inner diameter ID and an outer diameter OD₁. Correspondingly, surgical screw 50 with which guide sleeve 10 is to be used, is shown to have an enlarged screw head 52 having an outer diameter OD₂ and a slot 54 for engagement by a tool for rotation of the screw during insertion; the screw shank 56 has an outer diameter OD₃. Inner diameter ID of guide sleeve 10 is just larger than screw shank diameter OD₃ but is less than screw head diameter OD₂. Since the incision need only have a diameter sufficient to receive thereinto the guide sleeve, less trauma to the patient and faster healing both result.

In a variation, the inner diameter of the deformable sleeve is selected to match the diameter of the drill bit. This allows the drill bit to fit snugly and allow for more accurate drilling. The deformable drill sleeve will then deform as the threads of the screw and head pass through it, and will negate the need of a second, relatively rigid hollow sleeve.

Guide sleeve 10 is deformable and elastically, temporarily, locally expandable and is made of appropriate biocompatible elastic material. An example of such a sleeve would be surgical-grade stainless steel woven into a sleeve form supported by a trocar (not shown).

A second embodiment of guide sleeve 100 is shown in FIG. 2, identical dimensionally to guide sleeve 10 in FIG. 1 for a surgical screw 50. Guide sleeve 100 includes a longitudinally extending line of perforations 102 enabling the guide sleeve to split open as surgical screw 50 is inserted therethrough during implantation. Examples of materials useful for such a guide sleeve 100 include plastic materials such as polyethylene and polytetrafluoroethylene, which are known to be used in tearaway introducer sheaths for insertion of implantable catheters into the vasculature of a patient such as for use in hemodialysis, where the sheath is manually splittable to be removed from around the catheter after insertion of the catheter's distal portion into the blood vessel.

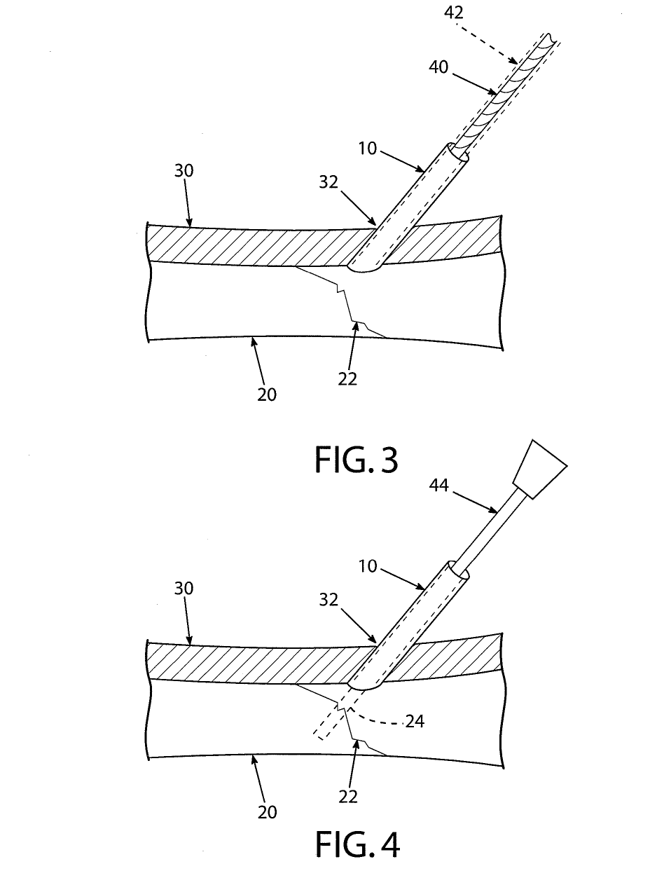

FIGS. 3 to 7 illustrate steps in a bone fixing procedure in which is used guide sleeve 10 (or, alternatively, guide sleeve 100) of the present invention and comprising the method of the present invention. Bone 20 has a fracture 22 in need of a surgical screw to secure the two bone components together to knit to each other. Soft tissue 30 of the patient overlies bone 20, with an incision 32 therethrough to expose a drill site.

FIG. 3 illustrates that a guide sleeve 10 is inserted through the incision 32 to the drill site, and is angled to match the angle at which a screw hole will be drilled; the general diameter of the incision 32 is substantially less than the diameter of screw head 52 and equal to the OD₁ of guide sleeve 10. Drill bit 40 of a surgical drill is shown inserted into guide sleeve 10 to drill a hole for the surgical screw that traverses the fracture 22. Alternatively, a rigid tube drill guide 42 (shown in dashed lines) may be used for immediately surrounding the drill bit, having an outer diameter less than the ID of the guide sleeve. In FIG. 4, a drilled hole 24 is shown in dashed lines, having been drilled using drill bit 40 of FIG. 3 which has now been removed, and a depth gauge 44 has been inserted through guide sleeve 10 to ascertain that a proper depth of drilled hole 24 has been attained. If the depth is insufficient, drill bit 40 may easily be re-inserted through guide sleeve 10 for additional drilling, until sufficient depth has been achieved.

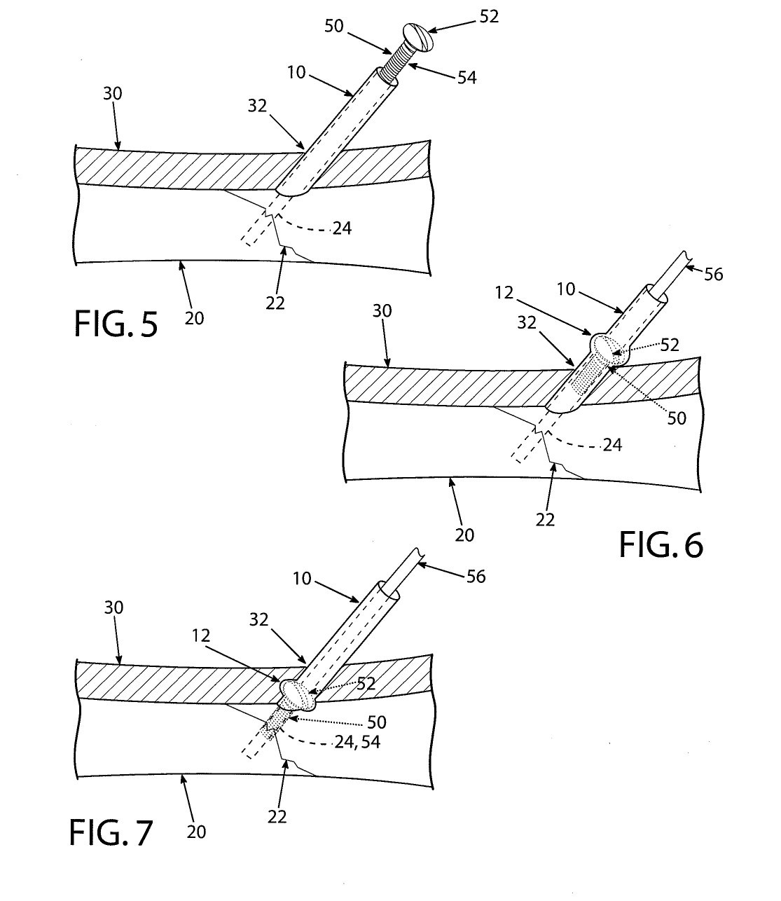

In FIG. 5, after drilled hole 24 has been created to desired depth, the drill bit and depth gauge have been removed and the surgical screw 50 has been positioned to be inserted into the exposed end of guide sleeve 10, with enlarged screw head 52 being larger than the ID of the guide sleeve. FIG. 6 shows the surgical screw 50 in dashed lines within guide sleeve 10 about to be threaded into the drilled hole 24. Using a rotatable tool such as a screw driver 56 to push and/or rotate the screw 50, enlarged screw head 52 is shown having been urged into the guide sleeve and having locally enlarged the guide sleeve at enlargement 12.

In FIG. 7, surgical screw 50 has been completely threaded into the drilled hole 24, thus holding both portions of the bone 20 together to become knitted. Enlarged screw head 52 has enlarged the portion of guide sleeve 10 adjacent the drill site at enlargement 12, and the temporarily stretched portions of the incision 32 proximal of enlargement 12 have resumed their previous dimensions, which were less than the diameter OD₂ of the screw head 52 and the consequent guide sleeve enlargement 12. Guide sleeve 10 may now be removed.

It will be appreciated by those skilled in the art that changes could be made to the embodiments described above without departing from the broad inventive concept thereof It is understood, therefore, that this invention is not limited to the particular embodiments disclosed, but it is intended to cover modifications within the spirit and scope of the present invention as defined by the appended claims.

What is claimed is:

1. A guide sleeve for guided insertion of a surgical screw therethrough to a site of a drilled hole through a fractured bone, comprising:
a length of tubing of elastic material temporarily enlargeable by passage therethrough of an enlarged head of a surgical screw during threaded insertion into a drilled hole, enabling a soft tissue incision smaller in diameter than the diameter of the enlarged screw head,
wherein the tubing has an inner diameter ID less than an outer diameter OD₂ of the surgical screw's enlarged head.

2. The guide sleeve of claim 1, wherein the length of tubing is a woven mesh of stainless steel.

3. The guide sleeve of claim 1, wherein the length of tubing includes longitudinally therealong a line of perforations enabling splitting of the tubing during screw insertion therethrough.

4. The guide sleeve of claim 3, wherein the length of tubing comprises plastic material.

5. A method of inserting a surgical screw into a drilled hole through a fractured bone, comprising the steps of:
providing a surgical screw having an enlarged head with an outer diameter OD₂;
providing a guide sleeve of elastic material comprising a length of tubing wherein the tubing has an inner diameter ID less than outer diameter OD₂ of the surgical screw's enlarged head;
forming a soft tissue incision at a surgical site to access the fractured bone and a hole drilled into the fractured bone thereat, the incision having a diameter only as large as the outer diameter of the guide sleeve and less than OD₂;
then, inserting the guide sleeve into the incision;
then, inserting the surgical screw into the guide sleeve and urging it therealong and therethrough whereby the enlarged screw head temporarily enlarges the diameter of the guide sleeve when passing therethrough, until the surgical screw is fully threaded into the drilled hole; and
then, removing the guide sleeve from the incision.

6. The method of claim 5, comprising the step, prior to screw insertion into the guide sleeve, inserting a drill bit through the guide sleeve and drilling the drilled hole into the fractured bone at the surgical site.

7. The method of claim 6, comprising the step, after drilling the drilled hole, of withdrawing the drill bit from the guide sleeve and inserting a depth gauge therethrough and into the drilled hole to ascertain the depth of the drilled hole.

8. The method of claim 7, if the drilled hole is not of sufficient depth, comprising the additional steps of removing the depth gauge from the guide sleeve, and re-inserting the drill bit through the guide sleeve and drilling the drilled hole to a sufficient depth.

9. The method of claim 5, comprising the step, prior to screw insertion into the guide sleeve, inserting a drill bit, surrounded by a rigid tube drill guide, through the guide sleeve and drilling the drilled hole into the fractured bone at the surgical site.
